(19) United States
(12) Patent Application Publication
(10) Pub. No.: US 2013/0267961 A1
(43) Pub. Date: Oct. 10, 2013

(54) IMPACTOR

(71) Applicants: Richard G. MAULDIN, Erie, CO (US); Vikas V. PATEL, Denver, CO (US); Scott A. YERBY, Montara, CA (US); Paul SAND, Redwood City, CA (US)

(72) Inventors: Richard G. MAULDIN, Erie, CO (US); Vikas V. PATEL, Denver, CO (US); Scott A. YERBY, Montara, CA (US); Paul SAND, Redwood City, CA (US)

(21) Appl. No.: 13/794,611

(22) Filed: Mar. 11, 2013

Related U.S. Application Data
(60) Provisional application No. 61/609,002, filed on Mar. 9, 2012.

(51) Int. Cl. A61B 17/88

(52) U.S. Cl. CPC A61B 17/8872
USPC 606/99

(57) ABSTRACT

This application relates generally to impactors. More specifically, this application relates to impactors used in medical procedures such as bone fixation or fusion. In some embodiments, the impactors include features that prevent or reduce the likelihood of over-insertion of an implant within a bone cavity.

### CROSS REFERENCE TO RELATED APPLICATIONS

This application claims priority to U.S. Provisional Application No. 61/609,002, filed Mar. 9, 2012, titled “IMPACTOR,” which is hereby incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference. For example, this application incorporates by reference in their entireties U.S. Patent Publication No. 2011/0087294 and U.S. Patent Publication No. 2011/0118785.

### FIELD

This application relates generally to impactors. More specifically, this application relates to impactors used in medical procedures such as bone fixation or fusion.

### BACKGROUND

Many types of hardware are available both for the fixation of bones that are fractured and for the fixation of bones that are to be fused (arthrodesed).

For example, the human hip girdle is made up of three large bones joined by three relatively immobile joints. One of the bones is called the sacrum and it lies at the bottom of the lumbar spine, where it connects with the L5 vertebra. The other two bones are commonly called “hip bones” and are technically referred to as the right ilium and-the left ilium. The sacrum connects with both hip bones at the sacroiliac joint (in shorthand, the SI-Joint).

The SI-Joint functions in the transmission of forces from the spine to the lower extremities, and vice-versa. The SI-Joint has been described as a pain generator for up to 22% of lower back pain.

To relieve pain generated from the SI Joint, sacroiliac joint fusion is typically indicated as surgical treatment, e.g., for degenerative sacroiliitis, inflammatory sacroiliitis, iatrogenic instability of the sacroiliac joint, osteitis condensans ilii, or traumatic fracture dislocation of the pelvis. Currently, screws and screws with plates are used for sacro-iliac fusion. At the same time the cartilage has to be removed from the “synovial joint” portion of the SI joint. This requires a large incision to approach the damaged, subluxed, dislocated, fractured, or degenerative joint.

An alternative implant that is not based on the screw design can also be used to fuse the SI-Joint. Such an implant can have a triangular cross-section, for example, as further described below. To insert the implant, a cavity can be formed into the bone, and the implant can then be inserted into the cavity using a tool such as an impactor. Over-insertion of the implant caused by striking the implant with too much force or striking the implant too many times can cause a variety of problems, such as loss of implant stability within the cavity. Therefore, an improved device and method for insertion of the implant is desirable.

### SUMMARY OF THE DISCLOSURE

This application relates generally to impactors. More specifically, this application relates to impactors used in medical procedures such as bone fixation or fusion. In some embodiments, the impactors include features that prevent or reduce the likelihood of over-insertion of an implant within a bone cavity.

In some embodiments, an impactor for driving an implant is provided. The impactor includes an elongate body having a proximal end, a distal end and a channel running therethrough; wherein the distal end includes a recess defined by a wall portion, the recess having a depth of between about 1 to 5 mm, the recess shaped to receive a proximal portion of the implant.

In some embodiments, the recess has a rectilinear transverse cross-section.

In some embodiments, the recess has a triangular transverse cross-section.

In some embodiments, the recess has a curvilinear transverse cross-section.

In some embodiments, the wall portion is continuous.

In some embodiments, the wall portion is formed by a plurality of discrete wall segments.

In some embodiments, the number of discrete wall segments corresponds to the number of sides of the implant.

In some embodiments, an implant for insertion into a bone cavity is provided. The implant includes an elongate body having a longitudinal axis, a rectilinear transverse cross-sectional profile, a distal end and a proximal end; and a stop feature located at the proximal end of the elongate body, the stop feature extending out radially from the rectilinear cross-sectional profile of the elongate body, the stop feature having a diameter greater than the diameter of the bone cavity.

In some embodiments, the stop feature comprises one or more spikes that extend distally from the stop features, wherein the spikes are configured to penetrate the bone surrounding the bone cavity when the implant is inserted into the bone cavity.

In some embodiments, the spikes are barbed.

In some embodiments, the stop feature is integral with the elongate body.

In some embodiments, the stop feature is removably attached to the elongate body.

In some embodiments, an impactor for driving an implant is provided. The impactor includes an elongate body having a proximal end, a distal end and a channel running therethrough; wherein the distal end includes a recess defined by a wall portion comprising a plurality of sides joined at a plurality of apices, the recess having a rectilinear transverse cross-section, the recess shaped to receive a proximal portion of the implant.

In some embodiments, at least one of the apices has a cutout that provides access to the recess.

In some embodiments, the impactor further includes an O-ring disposed around the distal end and the cutout such that a portion of the O-ring extends into the recess.

In some embodiments, the distal end has a cross-sectional profile that matches the implant's cross-sectional profile.

In some embodiments, the proximal end forms a head portion having a cross-sectional profile that matches the cross-sectional profile of the distal end.

In some embodiments, the proximal end forms a head portion having a cross-sectional profile that is greater in size than the cross-sectional profile of the distal end.

In some embodiments, the head portion comprises a plurality of slots that are arranged in a configuration that matches the profile of the distal end.

In some embodiments, the impactor further includes at least one cantilevered leaf spring that extends into the recess.

In some embodiments, a method of inserting an implant into a bone cavity. The method includes providing an impactor having an elongate body with a proximal end, a distal end and a channel running therethrough, the distal end including a recess having a depth of between about 1 to 5 mm, the recess shaped to receive a proximal portion of the implant; fitting the proximal portion of the implant into the recess of the impactor; driving the implant into the bone cavity using the impactor until the distal end of the impactor abuts against the bone surrounding the bone cavity; and leaving the proximal portion of the implant to extend about 1 to 5 mm above the bone surrounding the bone cavity.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 illustrates an embodiment of an implant structure.

FIGS. 2A-2D are side section views of the formation of a broached bore in bone according to one embodiment of the invention.

FIGS. 2E and 2F illustrate the assembly of a soft tissue protector system for placement over a guide wire.

FIGS. 3 and 4 are, respectively, anterior and posterior anatomic views of the human hip girdle comprising the sacrum and the hip bones (the right ilium, and the left ilium), the sacrum being connected with both hip bones at the sacroiliac joint (in shorthand, the SI-Joint).

FIGS. 5 to 7A and 7B are anatomic views showing, respectively, a pre-implanted perspective, implanted perspective, implanted anterior view, and implanted cranio-caudal section view, the implantation of three implant structures for the fixation of the SI-Joint using a lateral approach through the ilium, the SI-Joint, and into the sacrum.

FIG. 8A is a side view of one embodiment of an impactor with a cupped end engaged with an implant.

FIG. 8B is a close up side view of the cupped end of the impactor of FIG. 8A.

FIG. 8C is an end view of one embodiment of an impactor with a cupped end.

FIG. 9A is a perspective view of another embodiment of an impactor with a cupped end having a plurality of wall portions.

FIG. 9B is an end view of another embodiment of an impactor with a cupped end having a plurality of wall portions.

FIG. 10 is a side view of an embodiment of an implant with a stop feature.

FIGS. 11A-11D illustrate another embodiment of an impactor with a cupped distal end.

FIGS. 11E-11N illustrate embodiments of an implant holding mechanism that includes one or more cantilevered leaf springs.

FIGS. 12A-12C illustrate yet another embodiment of an impactor with a cupped distal end.

### DETAILED DESCRIPTION

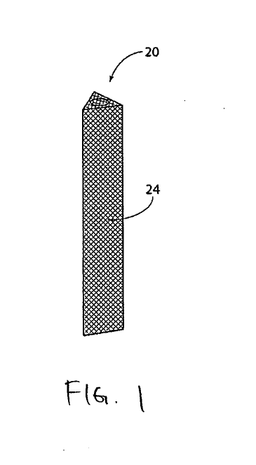

Elongated, stem-like implant structures 20 like that shown in FIG. 1 make possible the fixation of the SI-Joint (shown in anterior and posterior views, respectively, in FIGS. 3 and 4) in a minimally invasive manner. These implant structures 20 can be effectively implanted through the use a lateral surgical approach. The procedure is desirably aided by conventional lateral, inlet, and outlet visualization techniques, e.g., using X-ray image intensifiers such as a C-arms or fluoroscopes to produce a live image feed, which is displayed on a TV screen.

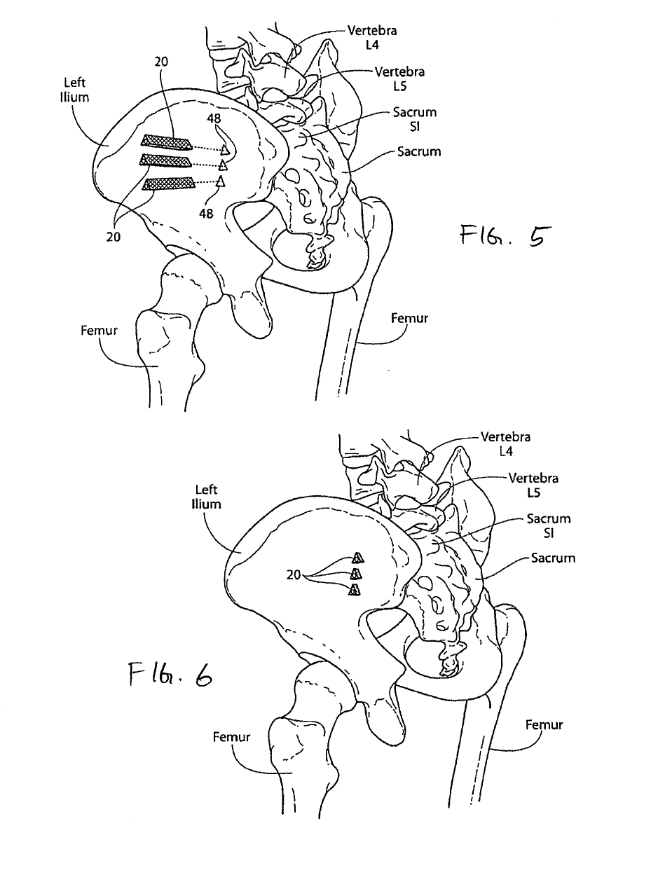

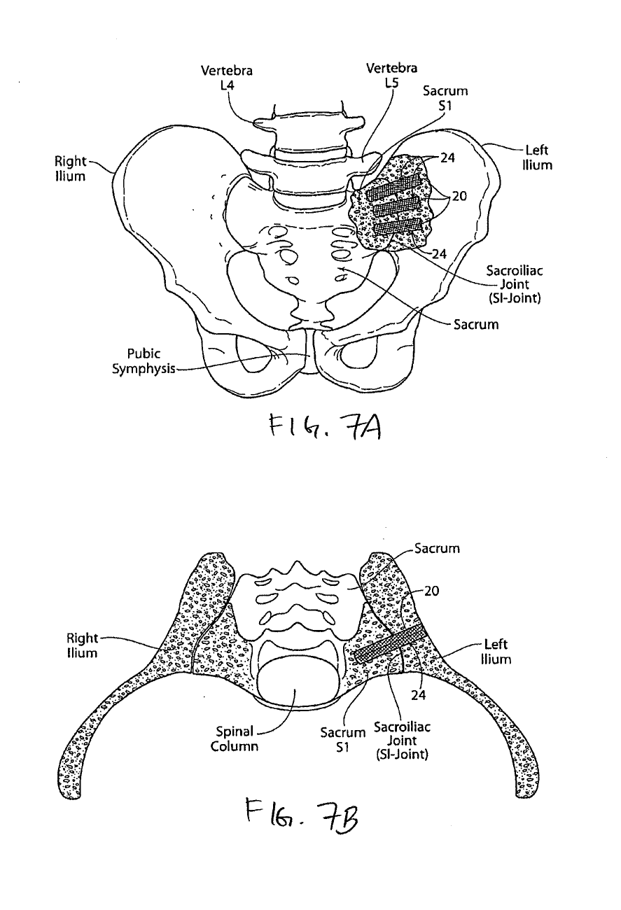

In one embodiment of a lateral approach (see FIGS. 5, 6, and 7A/B), one or more implant structures 20 are introduced laterally through the ilium, the SI-Joint, and into the sacrum. This path and resulting placement of the implant structures 20 are best shown in FIGS. 6 and 7A/B. In the illustrated embodiment, three implant structures 20 are placed in this manner. Also in the illustrated embodiment, the implant structures 20 are rectilinear in cross section and triangular in this case, but it should be appreciated that implant structures 20 of other rectilinear cross sections can be used. For example, the implant structures can have a square cross-section. In some embodiments, the implant structures can have a curvilinear cross-section, such as circular, oval or elliptical. The cross-sections discussed above refer to the transverse cross-section of the implant rather than a longitudinal cross-section taken along the longitudinal axis of the implant structure. In addition, the term rectilinear describes a device that is defined or substantially defined by straight lines. This includes, for example, triangles, squares, and other polygons, and also includes triangles, squares and other polygons having rounded corners. In contrast, the term curvilinear is meant to describe devices that are defined by only curved lines, such as a circle or ellipse, for example.

Before undertaking a lateral implantation procedure, the physician identifies the SI-Joint segments that are to be fixated or fused (arthrodesed) using, e.g., the Fortin finger test, thigh thrust, FABER, Gaenslen's, compression, distraction, and diagnostic SI joint injection.

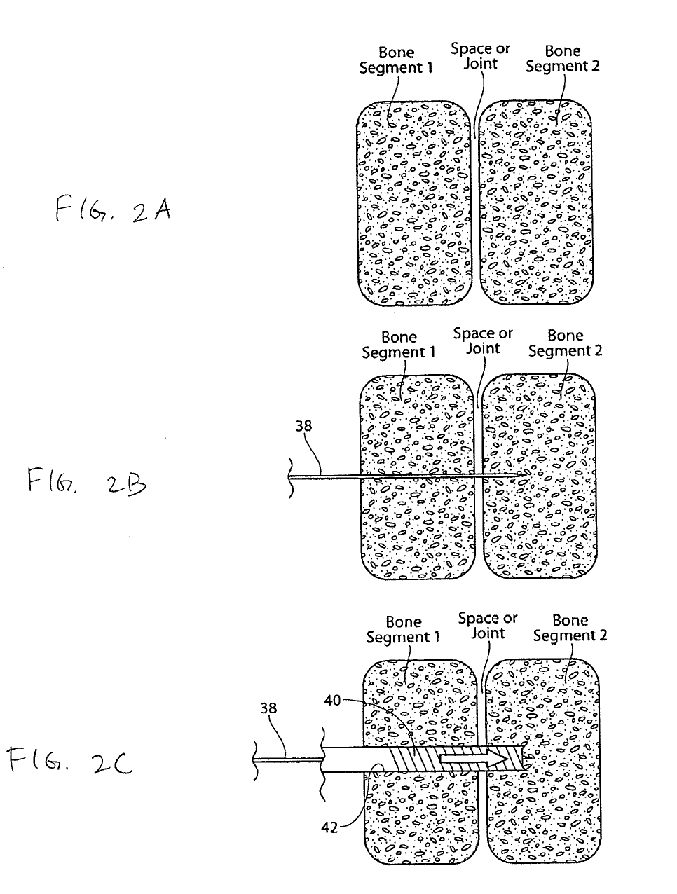

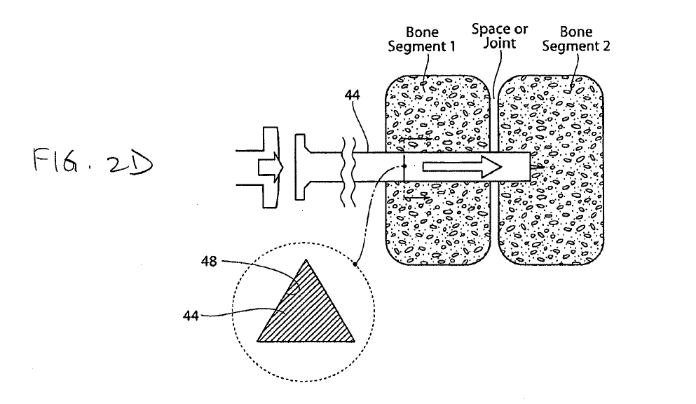

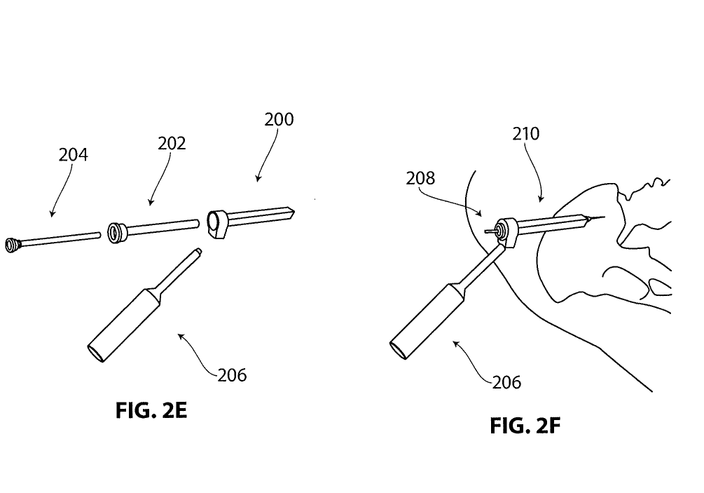

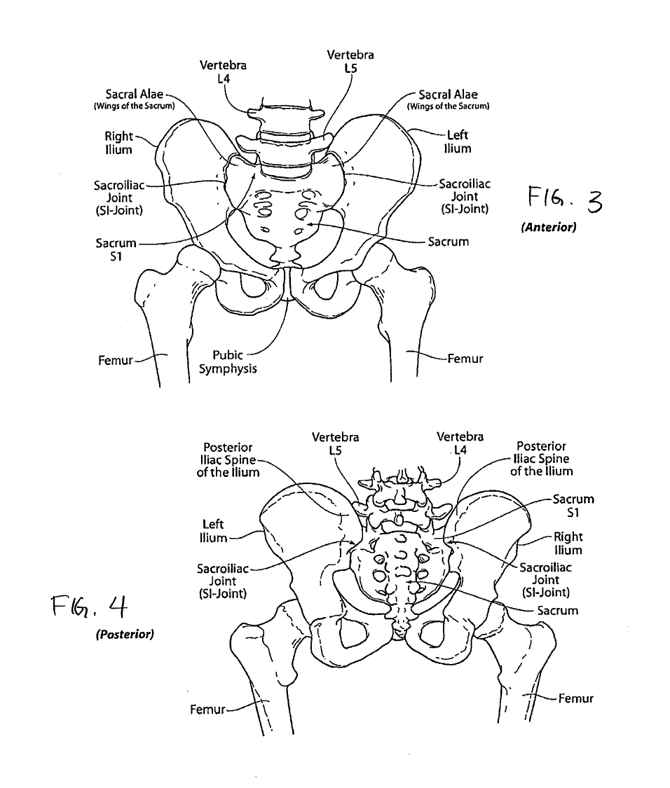

Aided by lateral, inlet, and outlet C-arm views, and with the patient lying in a prone position, the physician aligns the greater sciatic notches and then the alae (using lateral visualization) to provide a true lateral position. A 3 cm incision is made starting aligned with the posterior cortex of the sacral canal, followed by blunt tissue separation to the ilium. From the lateral view, the guide pin 38 (with sleeve (not shown)) (e.g., a Steinmann Pin) is started resting on the ilium at a position inferior to the sacrum end plate and just anterior to the sacral canal. In the outlet view, the guide pin 38 should be parallel to the sacrum end plate at a shallow angle anterior (e.g., 15.degree. to 20.degree. off the floor, as FIG. 7A shows). In a lateral view, the guide pin 38 should be posterior to the sacrum anterior wall. In the outlet view, the guide pin 38 should be superior to the first sacral foramen and lateral of mid-line. This corresponds generally to the sequence shown diagrammatically in FIGS. 2A and 2B. A soft tissue protector (not shown) is desirably slipped over the guide pin 38 and firmly against the ilium before removing the guide pin sleeve (not shown).

Over the guide pin 38 (and through the soft tissue protector), the pilot bore 42 is drilled in the manner previously described, as is diagrammatically shown in FIG. 2C. The pilot bore 42 extends through the ilium, through the SI-Joint, and into the 51. The drill bit 40 is removed.

The shaped broach 44 is tapped into the pilot bore 42 over the guide pin 38 (and through the soft tissue protector) to create a broached bore 48 with the desired profile for the implant structure 20, which, in the illustrated embodiment, is triangular. This generally corresponds to the sequence shown diagrammatically in FIG. 2D. The triangular profile of the broached bore 48 is also shown in FIG. 5.

FIGS. 2E and 2F illustrate an embodiment of the assembly of a soft tissue protector or dilator or delivery sleeve 200 with a drill sleeve 202, a guide pin sleeve 204 and a handle 206. In some embodiments, the drill sleeve 202 and guide pin sleeve 204 can be inserted within the soft tissue protector 200 to form a soft tissue protector assembly 210 that can slide over the guide pin 208 until bony contact is achieved. The soft tissue protector 200 can be any one of the soft tissue protectors or dilators or delivery sleeves disclosed herein. In some embodiments, an expandable dilator or delivery sleeve 200 as disclosed herein can be used in place of a conventional soft tissue dilator. In the case of the expandable dilator, in some embodiments, the expandable dilator can be slid over the guide pin and then expanded before the drill sleeve 202 and/or guide pin sleeve 204 are inserted within the expandable dilator. In other embodiments, insertion of the drill sleeve 202 and/or guide pin sleeve 204 within the expandable dilator can be used to expand the expandable dilator.

In some embodiments, a dilator can be used to open a channel though the tissue prior to sliding the soft tissue protector assembly 210 over the guide pin. The dilator(s) can be placed over the guide pin, using for example a plurality of sequentially larger dilators or using an expandable dilator. After the channel has been formed through the tissue, the dilator(s) can be removed and the soft tissue protector assembly can be slid over the guide pin. In some embodiments, the expandable dilator can serve as a soft tissue protector after being expanded. For example, after expansion the drill sleeve and guide pin sleeve can be inserted into the expandable dilator.

As shown in FIGS. 5 and 6, a triangular implant structure 20 can be now tapped through the soft tissue protector over the guide pin 38 through the ilium, across the SI-Joint, and into the sacrum, until the proximal end of the implant structure 20 is flush against the lateral wall of the ilium (see also FIGS. 7A and 7B). The guide pin 38 and soft tissue protector are withdrawn, leaving the implant structure 20 residing in the broached passageway, flush with the lateral wall of the ilium (see FIG. 7A and 7B). In the illustrated embodiment, two additional implant structures 20 are implanted in this manner, as FIG. 6 best shows. In other embodiments, the proximal ends of the implant structures 1020 are left proud of the lateral wall of the ilium, such that they extend 1, 2, 3 or 4 mm outside of the ilium. This ensures that the implants 20 engage the hard cortical portion of the ilium rather than just the softer cancellous portion, through which they might migrate if there was no structural support from hard cortical bone. The hard cortical bone can also bear the loads or forces typically exerted on the bone by the implant 20.

The implant structures 20 are sized according to the local anatomy. For the SI-Joint, representative implant structures 20 can range in size, depending upon the local anatomy, from about 35 mm to about 60 mm in length, and about a 7 mm inscribed diameter (i.e. a triangle having a height of about 10.5 mm and a base of about 12 mm). The morphology of the local structures can be generally understood by medical professionals using textbooks of human skeletal anatomy along with their knowledge of the site and its disease or injury. The physician is also able to ascertain the dimensions of the implant structure 20 based upon prior analysis of the morphology of the targeted bone using, for example, plain film x-ray, fluoroscopic x-ray, or MRI or CT scanning.

Using a lateral approach, one or more implant structures 20 can be individually inserted in a minimally invasive fashion across the SI-Joint, as has been described. Conventional tissue access tools, obturators, cannulas, and/or drills can be used for this purpose. Alternatively, the novel tissue access tools described above and in co-pending U.S. Application No. 61/609,043, titled “TISSUE DILATOR AND PROTECTER” and filed Mar. 9, 2012, can also be used. No joint preparation, removal of cartilage, or scraping are required before formation of the insertion path or insertion of the implant structures 20, so a minimally invasive insertion path sized approximately at or about the maximum outer diameter of the implant structures 20 can be formed.

The implant structures 20 can obviate the need for autologous bone graft material, additional pedicle screws and/or rods, hollow modular anchorage screws, cannulated compression screws, threaded cages within the joint, or fracture fixation screws. Still, in the physician's discretion, bone graft material and other fixation instrumentation can be used in combination with the implant structures 20.

In a representative procedure, one to six, or perhaps up to eight, implant structures 20 can be used, depending on the size of the patient and the size of the implant structures 20. After installation, the patient would be advised to prevent or reduce loading of the SI-Joint while fusion occurs. This could be about a six to twelve week period or more, depending on the health of the patient and his or her adherence to post-op protocol.

The implant structures 20 make possible surgical techniques that are less invasive than traditional open surgery with no extensive soft tissue stripping. The lateral approach to the SI-Joint provides a straightforward surgical approach that complements the minimally invasive surgical techniques. The profile and design of the implant structures 20 minimize or reduce rotation and micromotion. Rigid implant structures 20 made from titanium provide immediate post-op SI Joint stability. A bony in-growth region 24 comprising a porous plasma spray coating with irregular surface supports stable bone fixation/fusion. The implant structures 20 and surgical approaches make possible the placement of larger fusion surface areas designed to maximize post-surgical weight bearing capacity and provide a biomechanically rigorous implant designed specifically to stabilize the heavily loaded SI-Joint.

To improve the stability and weight bearing capacity of the implant, the implant can be inserted across three or more cortical walls. For example, after insertion the implant can traverse two cortical walls of the ilium and at least one cortical wall of the sacrum. The cortical bone is much denser and stronger than cancellous bone and can better withstand the large stresses found in the SI-Joint. By crossing three or more cortical walls, the implant can spread the load across more load bearing structures, thereby reducing the amount of load borne by each structure. In addition, movement of the implant within the bone after implantation is reduced by providing structural support in three locations around the implant versus two locations.

When driving the implant into a cavity formed in the bone, it can be possible to inadvertently insert the implant too far such that the implant loses support from the outermost, i.e. the most lateral, cortical wall, leaving the implant supported by only two cortical walls and thereby compromising joint stability.

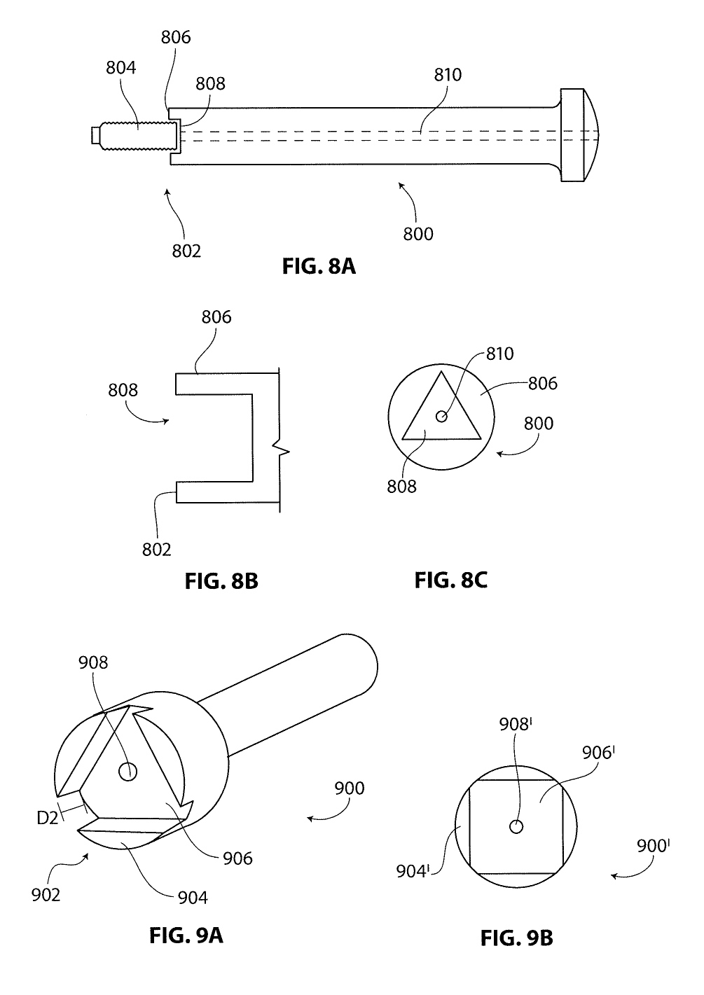

As illustrated in FIGS. 8A-8C, an embodiment of an impactor 800 having a cupped or recessed distal end 802 can be used to prevent or reduce the likelihood of over-insertion of the implant 804. The distal end 802 can include a wall portion 806 that defines a recess 808 that is shaped to receive the proximal end of the implant 804. For example, the recess 808 can be triangular to receive an implant 804 with a triangular cross-section. In addition, the impactor 800 can include a channel 810 for receiving a guide pin (not shown) that extends through the longitudinal axis of the impactor 800.

The impactor 800 can be used to drive the implant 804 into a bone cavity created by, for example, a drill and broach. The cavity is generally just large enough to receive the implant 804. In some embodiments, the impactor 800 has a larger diameter and/or cross-sectional area than both the cavity and the implant 804, which prevents the impactor 800 from inadvertently entering the bone cavity and driving the implant 804 too far. Instead, the wall portion 806 of the distal end 802 of the impactor 800, which covers the proximal portion of the implant 804, eventually abuts against the bone surrounding the cavity during the insertion procedure, which stops forward progress of the impactor 800 and indicates to the operator than insertion of the implant 804 is complete. In other embodiments, at least one portion of the distal end 802 of the impactor 802 is not aligned with the bone cavity during the implant 804 insertion procedure, and eventually abuts against the bone surrounding the cavity when the implant 804 is fully inserted. In some embodiments, the wall portion 806 can be continuous and can completely enclose the proximal portion of the implant 804. In other embodiments, the wall portion 806 can be discontinuous and only covers a portion of the proximal portion of the implant 804.

The implant 804 can be left proud, i.e. projecting above the bone surface, to ensure that the implant 804 is fully supported by the outer or most proximal cortical wall. As illustrated in FIGS. 8A and 8B, the depth Dl of the recess 808 in the distal end 802 of the impactor 800 controls how much the implant 804 projects above the bone surface. In some embodiments, D1 can be between about 1 to 5 mm, 2 to 4 mm, or 2 to 3 mm. For example, an impactor 800 with a recess 808 having a depth of 2 mm can leave the implant 800 projecting about 2 mm from the bone surface.

In some embodiments, as illustrated in FIG. 9A, the impactor 900 can have a cupped distal end 902 that is formed from a plurality of wall portions 904. The plurality of wall portions 904 define a recess 906 having a depth D2, which in some embodiments can be between about 1 to 5 mm, 2 to 4 mm, or 2 to 3 mm. In some embodiments, the number of wall portions 904 can correspond to the number of sides of the implant. For example, an impactor 900 with three wall portions 904 that are angled at about 60 degrees to each other can be used with a triangular implant having three sides. The wall portions 904 function to secure the implant within the recess 906. In addition, the impactor 900 can have a channel 908 for receiving a guide pin that runs along the longitudinal axis of the impactor 900. During insertion of the implant, the wall portions 904 are designed to abut against the bone surrounding the bone cavity when the implant is fully inserted, leaving the implant proud as described above.

Similarly, FIG. 9B illustrates an embodiment of an impactor 900′ with a cupped end with four distal portions 904′ that are angled about 90 degrees to each other. This embodiment of the impactor 900′ can be used with a square or rectangular implant. Other embodiments of the impactor can include additional wall portions for use with implants with additional sides.

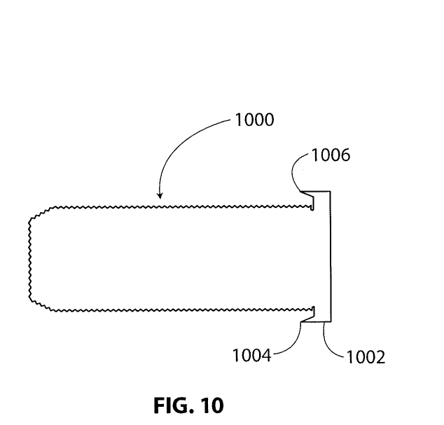

In other embodiments, as illustrated in FIG. 10, the implant 1000 itself can include a stop feature 1002 at the proximal end of the implant 1000. The stop feature 1002 projects outward radially or transversely and stops over-insertion of the implant 1000. The stop feature 1002 is designed to abut against the bone surrounding the bone cavity that receives the implant 1000. In some embodiments, the stop feature 1002 can include one or more spikes 1004 that can penetrate the bone surrounding the bone cavity to secure the implant 1000 in place and reduce both rotational movement and translational movement of the implant 1000 within the bone cavity. In some embodiments, the spikes 1004 can have barbs 1006 that further reduce the translational movement of the implant 100 within the bone cavity.

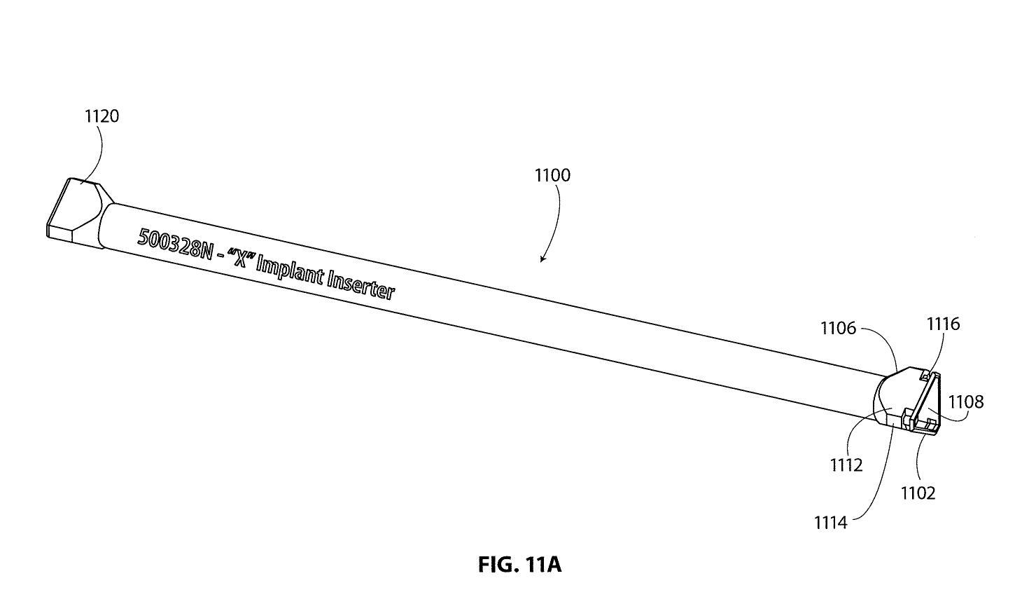

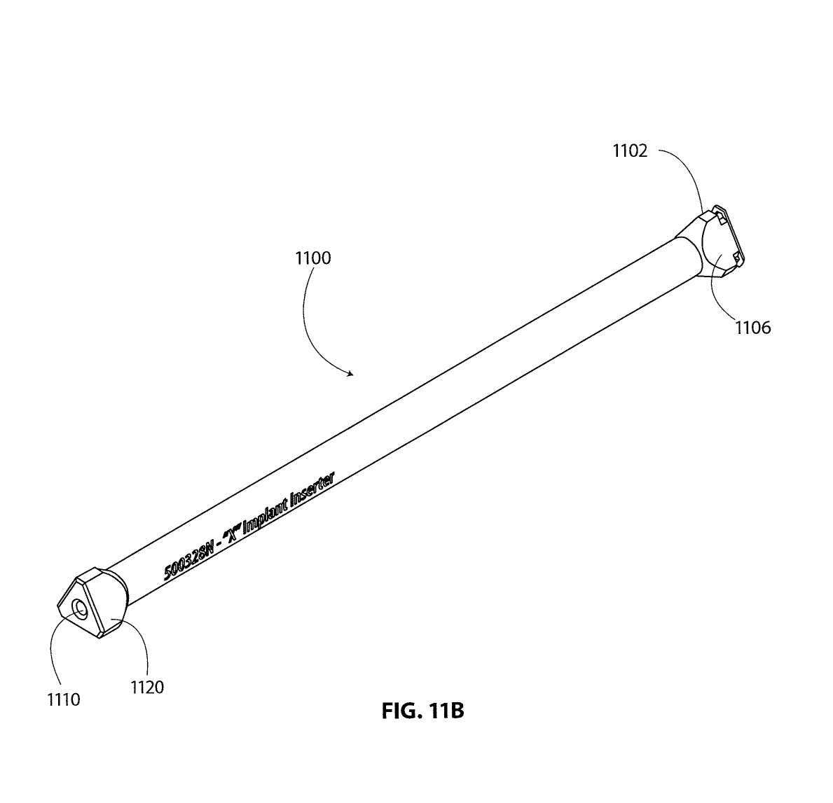

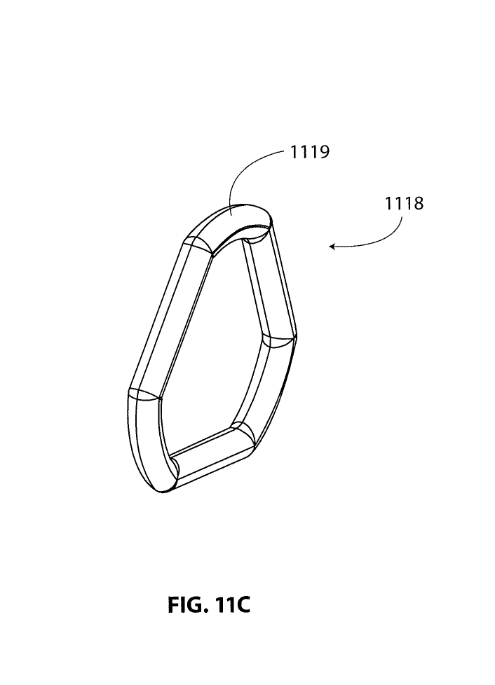

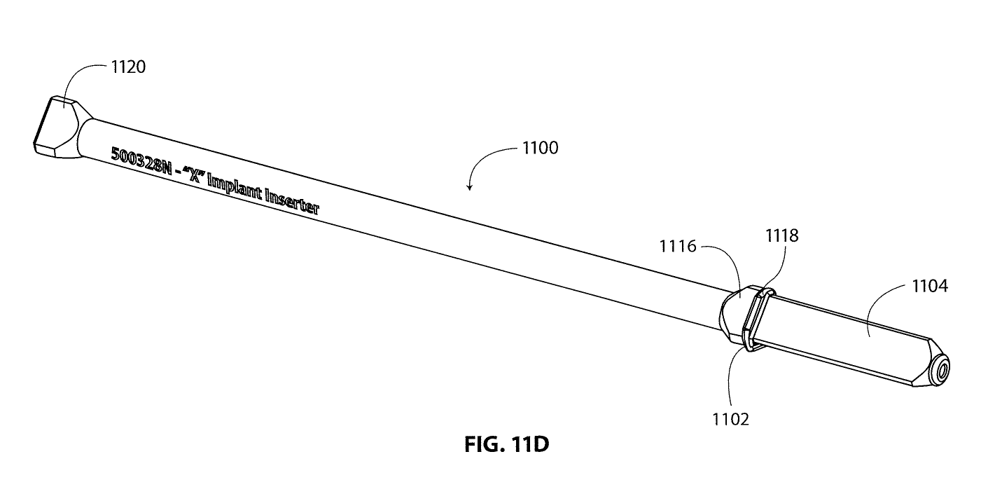

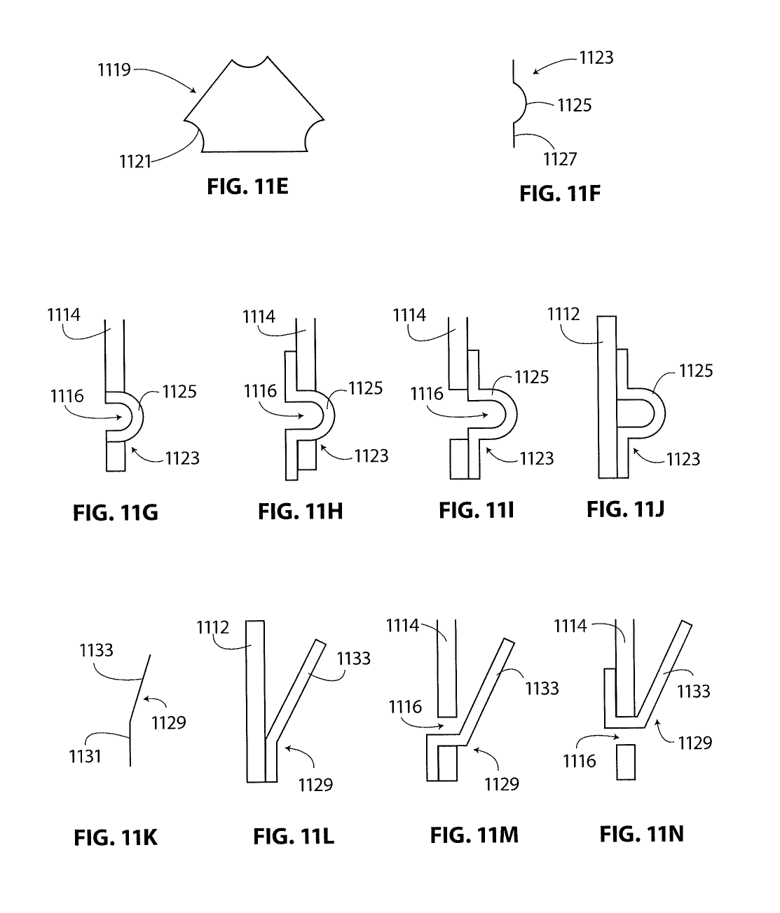

FIGS. 11A-11D illustrate another embodiment of an impactor 1100 having a cupped or recessed distal end 1102 that can be used to insert the implant 1104 into a bone cavity while preventing or reducing the likelihood of over-insertion of the implant 1104. The distal end 1102 can include a wall portion 1106 that defines a recess 1108 that is shaped to receive the proximal end of the implant 1104. For example, the recess 1108 can be triangular to receive an implant 1104 with a triangular cross-section, or another rectilinear shape to receive a rectilinear implant, or curvilinear to receive a curvilinear implant. In addition, the impactor 1100 can include a channel 1110 for receiving a guide pin (not shown) that extends through the longitudinal axis of the impactor 1100.

In some embodiments, the cupped distal end 1102 can have a wall portion 1106 formed from a plurality of planar wall sections 1112 that are joined at a plurality of apices 1114 to define a recess 1108 having a rectilinear transverse cross-section. For reference, the rectilinear cross-section is transverse to the longitudinal axis of the impactor. This configuration can provide the cupped distal end 1102 with a similar transverse cross-sectional profile as the implant, and can allow the impactor 1100 to be inserted through a rectilinear dilator that also has a matching transverse cross-sectional profile. For example, for insertion of a triangular implant, a triangular broach inserted through a triangular dilator can be used to form the cavity. Once the triangular cavity is formed, the broach can be removed and the impactor 1100 with a triangular cupped distal end 1102 and associated implant 1104 can be inserted through the triangular dilator. By maintaining the same cross-sectional profile between each device, the same dilator can be used to facilitate the passage of various devices through the patient's soft tissue, thereby reducing the number of different dilators that must be used in the procedure.

In some embodiments, the apices 1114 can each have a cutout 1116 at the same axial location of the impactor 1100 that provides access to the interior of the recess 1108 from the outside. In some embodiments, at least one of the apices 1114 has a cutout 1116. A band or O-ring 1118 can be placed around the cutouts 1116 and exterior of the cupped distal end 1102 such that a portion of the band or O-ring 1118 extends into the recess 1108 at each cutout 1116. The band or O-ring 1118 can be made from an elastic material such as rubber, silicon, plastic, or another polymeric material, that functions to reversibly grip and secure the implant 1104 to the impactor 1100 when the implant 1104 is inserted into the cupped distal end 1102. The band or O-ring can have profile that generally matches the cross-sectional profile of the cupped distal end 1102, except that the apices 1119 of the band or O-ring can be rounded off to a greater degree than the cupped distal end 1102 so that the band or O-ring can be secured within the cutouts 1116. Alternatively, a circular O-ring may be used that assumes the shape shown in FIG. 11C when it is stretched over the distal end of the impactor 1100. The gripping force exerted by the band or 0-ring on the implant 1104 is much less than the gripping force exerted by the bone cavity on the implant 1104 after insertion, which allows the impactor 1100 to simply be pulled away and separated from the implant 1104 after insertion of the implant 1104 into the bone cavity.

In some embodiments, as illustrated in FIGS. 11E-11L, the implant holding mechanism can be a cantilevered leaf spring 1119 instead of an O-ring. In some embodiments as illustrated in FIG. 11E, the leaf spring 1119 can be constructed in a shape that matches the transverse cross-sectional profile of the implant but with inwardly projecting arches 1121 that fit through the cutout 1116 and extend into the recess and function to grip an implant inserted into the recess with a gripping force that is much less than the gripping force exerted by the bone cavity on the implant 1104 after insertion. In some embodiments, the inwardly projecting arches 1121 can be partially hemisperically shaped so that the inwardly projecting arches 1121 present a sloping surface towards the implant as the implant is inserted into the recess. Alternatively, as illustrated in FIGS. 11F-11J, discrete cantilevered leaf springs 1123 that have an arched portion 1125 and one or more attachment portions 1127 can be disposed within the cutout 1116 to extend into the recess or can be disposed anywhere within the recess and against one or more wall sections 1112 such that the arched portion 1125 extends into the recess. The discrete leaf springs 1123 can be oriented longitudinally with the longitudinal axis of the impactor such that the implant pushes against a sloped surface of the arched portion of the leaf spring when the implant is inserted into the recess. The discrete leaf springs 1123 also function to grip an implant inserted into the recess with a gripping force that is much less than the gripping force exerted by the bone cavity on the implant 1104 after insertion. In some embodiments, the leaf spring(s) can be made of the same material as the impactor, such as stainless steel, titanium, or a metal alloy. In other embodiments, the leaf spring(s) can be made of a plastic or polymer material. Other embodiments of the cantilevered leaf spring are illustrated in FIGS. 11K-11N. The leaf spring 1129 can have an attachment portion 1131 and a lever portion 1133 that extends inwardly into the recess. The leaf spring 1129 can be positioned anywhere within the recess and against the wall section in a longitudinal orientation or can be positioned through a cutout to extend into the recess to present a sloping surface towards the implant.

The proximal end of the impactor 1100 can terminate in a head portion 1120 that has an impact or striking surface for an impacting device such as a slap hammer or mallet, for example, that can be used to drive the implant 1104 into the bone cavity. In some embodiments, the head portion 1120 can have a similar or the same transverse cross-sectional profile as the cupped distal end 1102. Such a configuration allows the entire impactor 1100 to be inserted through dilator having the same cross-sectional profile, and can also help the operator with aligning the impactor 1100 by making sure the head portion 1120 is aligned with the cupped distal end 1102 when viewed axially. In some embodiments, the impactor 1100 can be used without a dilator and the head portion 1120 can be used to estimate, align and/or adjust the implant's rotational alignment.

The impactor 1100 can be used to drive the implant 1104 into a bone cavity created by, for example, a drill and broach. The cavity is generally just large enough to receive the implant 1104. In some embodiments, the impactor 1100 has a larger diameter and/or cross-sectional area than both the cavity and the implant 1104, which prevents the impactor 1100 from inadvertently entering the bone cavity and driving the implant 1104 too far. The depth of the recess 1108 allows the implant 1104 to be left proud of the bone by a predetermined length, as described above.

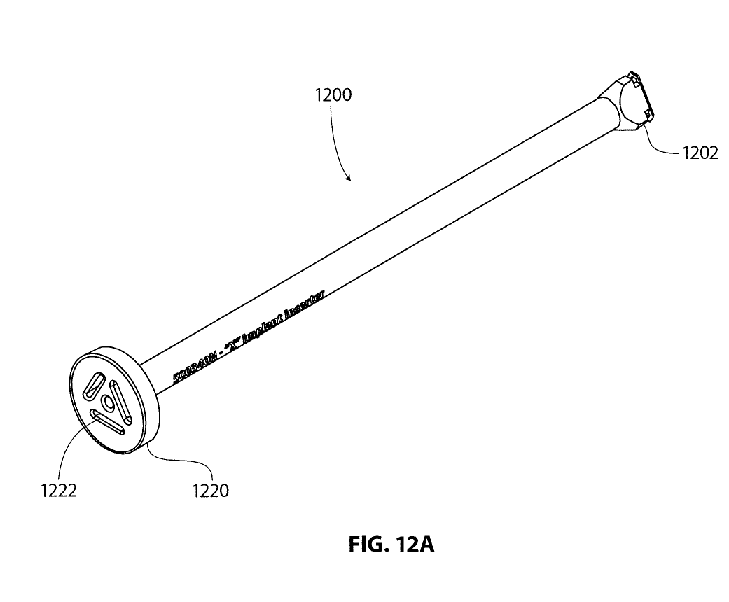

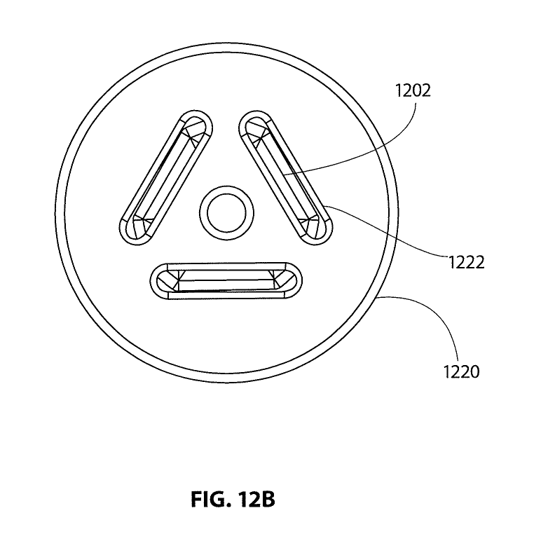

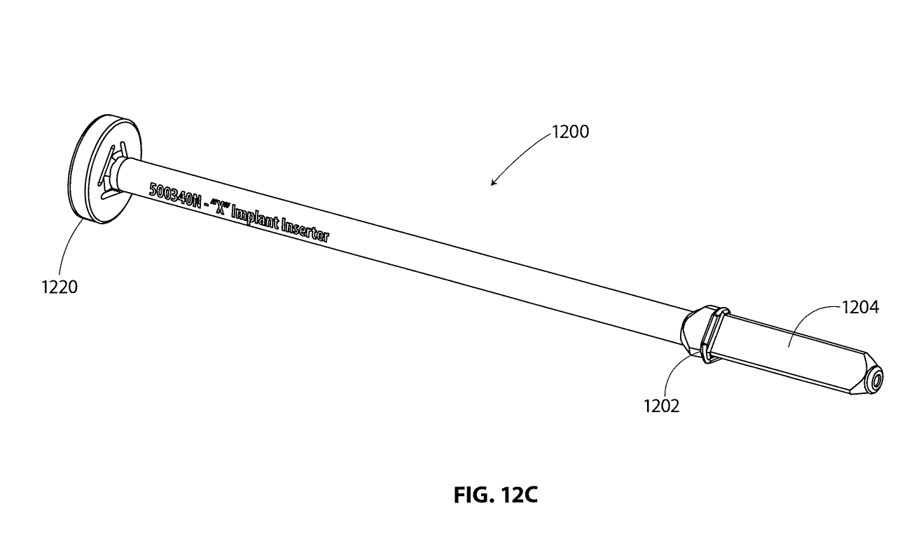

FIGS. 12A-12C illustrate another embodiment of an impactor 1200 having a cupped distal end 1202 that is similar to the impactor illustrated in FIGS. 11A-11D, except for the head portion. The head portion 1220 in this embodiment can have a different shape than the cupped distal end 1202. For example, the head portion 1220 can have a greater diameter than the cupped distal end 1202 such that the head portion 1220 cannot enter or pass through the dilator, which allows the head portion 1220 to also function as a stop that limits or controls the depth of penetration of the implant 1204 into the bone cavity. The head portion 1220 can have a flat striking surface and can be circular, curvilinear, or rectilinear. As illustrated, the head portion 1220 is circular. The head portion 1220 can also have a plurality of slots 1222 that are arranged to match the outer profile of the cupped distal end 1202. For example, for a triangular cupped distal end 1202, the head portion 1220 can have three slots 1222 that are arranged in a matching triangle. The slots 1222 assist the operator in aligning the impactor 1220 by visually aligning the cupped distal end 1202 with the slots 1222 when viewed axially.

Variations and modifications of the devices and methods disclosed herein will be readily apparent to persons skilled in the art. As such, it should be understood that the foregoing detailed description and the accompanying illustrations, are made for purposes of clarity and understanding, and are not intended to limit the scope of the invention, which is defined by the claims appended hereto. Any feature described in any one embodiment described herein can be combined with any other feature of any of the other embodiment whether preferred or not.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

What is claimed is:

1. An impactor for driving an implant, the impactor comprising:
an elongate body having a proximal end, a distal end and a channel running therethrough;
wherein the distal end includes a recess defined by a wall portion, the recess having a depth of between about 1 to 5 mm, the recess shaped to receive a proximal portion of the implant.

2. The impactor of claim 1, wherein the recess has a rectilinear transverse cross-section.

3. The impactor of claim 1, wherein the recess has a triangular transverse cross-section.

4. The impactor of claim 1, wherein the recess has a curvilinear transverse cross-section.

5. The impactor of claim 1, wherein the wall portion is continuous.

6. The impactor of claim 1, wherein the wall portion is formed by a plurality of discrete wall segments.

7. The impactor of claim 6, wherein the number of discrete wall segments corresponds to the number of sides of the implant.

8. An implant for insertion into a bone cavity, the implant comprising:
an elongate body having a longitudinal axis, a rectilinear transverse cross-sectional profile, a distal end and a proximal end; and
a stop feature located at the proximal end of the elongate body, the stop feature extending out radially from the rectilinear cross-sectional profile of the elongate body, the stop feature having a diameter greater than the diameter of the bone cavity.

9. The implant of claim 8, wherein the stop feature comprises one or more spikes that extend distally from the stop features, wherein the spikes are configured to penetrate the bone surrounding the bone cavity when the implant is inserted into the bone cavity.

10. The implant of claim 8, wherein the spikes are barbed.

11. The implant of claim 8, wherein the stop feature is integral with the elongate body.

12. The implant of claim 8, wherein the stop feature is removably attached to the elongate body.

13. An impactor for driving an implant, the impactor comprising:
an elongate body having a proximal end, a distal end and a channel running therethrough;
wherein the distal end includes a recess defined by a wall portion comprising a plurality of sides joined at a plurality of apices, the recess having a rectilinear transverse cross-section, the recess shaped to receive a proximal portion of the implant.

14. The impactor of claim 13, wherein at least one of the apices has a cutout that provides access to the recess.

15. The impactor of claim 14, further comprising an O-ring disposed around the distal end and the cutout such that a portion of the O-ring extends into the recess.

16. The impactor of claim 13, wherein the distal end has a cross-sectional profile that matches the implant's cross-sectional profile.

17. The impactor of claim 13, wherein the proximal end forms a head portion having a cross-sectional profile that matches the cross-sectional profile of the distal end.

18. The impactor of claim 13, wherein the proximal end forms a head portion having a cross-sectional profile that is greater in size than the cross-sectional profile of the distal end.

19. The impactor of claim 13, wherein the head portion comprises a plurality of slots that are arranged in a configuration that matches the profile of the distal end.

20. The impactor of claim 13, further comprising at least one cantilevered leaf spring that extends into the recess.

21. A method of inserting an implant into a bone cavity, the method comprising:
providing an impactor having an elongate body with a proximal end, a distal end and a channel running therethrough, the distal end including a recess having a depth of between about 1 to 5 mm, the recess shaped to receive a proximal portion of the implant;
fitting the proximal portion of the implant into the recess of the impactor;
driving the implant into the bone cavity using the impactor until the distal end of the impactor abuts against the bone surrounding the bone cavity; and
leaving the proximal portion of the implant to extend about 1 to 5 mm above the bone surrounding the bone cavity.
